(19) United States
(12) Patent Application Publication
(10) Pub. No.: US 2026/0340834 A1
(43) Pub. Date: Oct. 29, 2026

(54) MEDICAL IMAGE PROCESSING APPARATUS AND METHOD

(71) Applicant: Canon Kabushiki Kaisha, Tokyo (JP)

(72) Inventors: Ewan HEMINGWAY, Edinburgh (GB); Mark JENKINS, Edinburgh (GB)

(73) Assignee: Canon Kabushiki Kaisha, Tokyo (JP)

(21) Appl. No.: 19/188,081

(22) Filed: Apr. 24, 2025

(51) Int. Cl. G06T 7/11; A61B 6/50; G06T 5/60; G06T 7/136; G06T 15/08

(52) U.S. Cl. CPC G06T 7/11; G06T 5/60; G06T 7/136; G06T 15/08; A61B 6/501; A61B 6/507; G06T 2207/10016; G06T 2207/10024; G06T 2207/10081; G06T 2207/20081; G06T 2207/30016; G06T 2207/30056; G06T 2207/30101; G06T 2210/41

(57) ABSTRACT

A medical image processing apparatus comprising processing circuitry configured to acquire a plurality of sets of volumetric image data of an anatomical region of a patient or other subject, each set of acquired volumetric image data corresponding to a respective different first point in time, generate a segmentation of a region of interest of the anatomical region, generate a plurality of sets of intermediate volumetric image data, one or more of the plurality of sets of intermediate volumetric image data representing the region of interest at a different second point in time, each second point in time corresponding to a point in time between two or more first points in time, and generate an image sequence based on the plurality of sets of intermediate volumetric image data, the image sequence representing at least a part or all of the region of interest.

### FIELD

The present invention relates to a medical image processing apparatus and a medical image processing method.

### BACKGROUND

Multi-phase computer tomography (CT) is an imaging tool that may be used to assess perfusion of an organ or another anatomical structure of a patient or other subject. Multi-phase CT may allow for the generation of time resolved images of the organ or other anatomical structure. In order to assess the perfusion of an organ or another anatomical structure of the patient or other subject, a contrast material may be injected into the organ or anatomical structure and CT scans are performed at different times and/or different phases, after injection of the contrast material.

For example, when the organ comprises the brain, multi-phase CT may be used to assess the status of perfusion, such as the collateral flow, in the brain of a patient or another subject having a stroke or another medical condition. A patient with poor collateral flow may not benefit from some of the available treatments, such as revascularisation.

Multi-phase CT may allow for the detection of important physiology, such as a temporal delay in the filling of vessels, which may not be observable with a single-phase method, such as single-phase CT. A temporal resolution of multi-phase CT may be less compared to four-dimensional (4D) CT, in which a large number of frames may be acquired. For example, in 4D CT about twenty frames may be acquired, whereas in multi-phase CT about three frames may be acquired. The comparably low temporal resolution of multi-phase CT may make it difficult to assess the flow in and/or filling of vessels in the organ or other anatomical structure.

An availability of scanners capable to perform 4D CT scans at hospitals or medical centres may be limited, e.g. compared to multi-phase CT. Additionally, 4D CT may be more complex than other imaging techniques. As such, it may be desirable to improve the visualisation of perfusion and/or filling of vessels in the organ or other anatomical structure of the patient using multi-phase CT.

### DESCRIPTION

Embodiments are now described by way of non-limiting example with reference to the accompanying drawings in which:

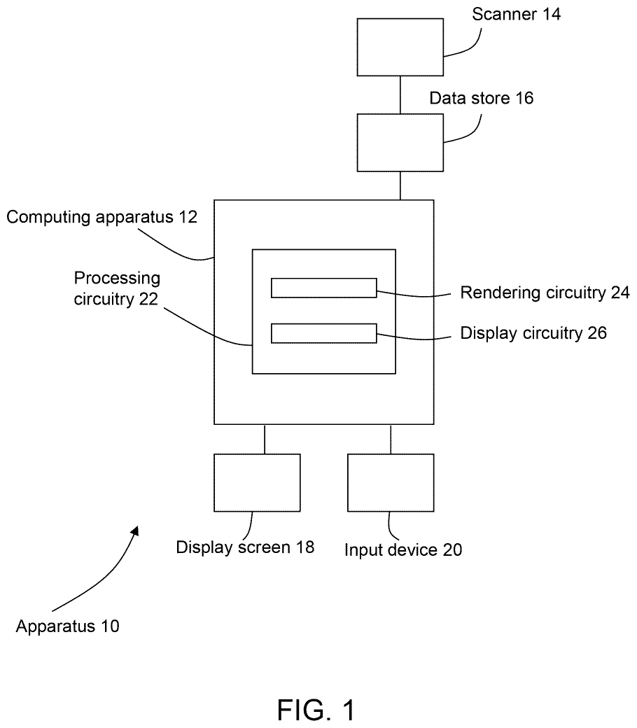

FIG. 1 is a schematic illustration of a medical image processing apparatus according to an embodiment;

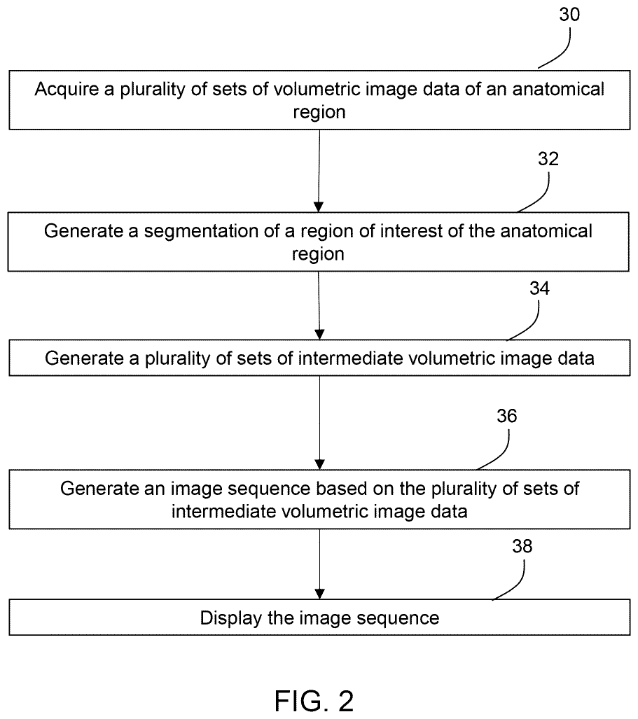

FIG. 2 is a flow chart illustrating in overview a process of an embodiment;

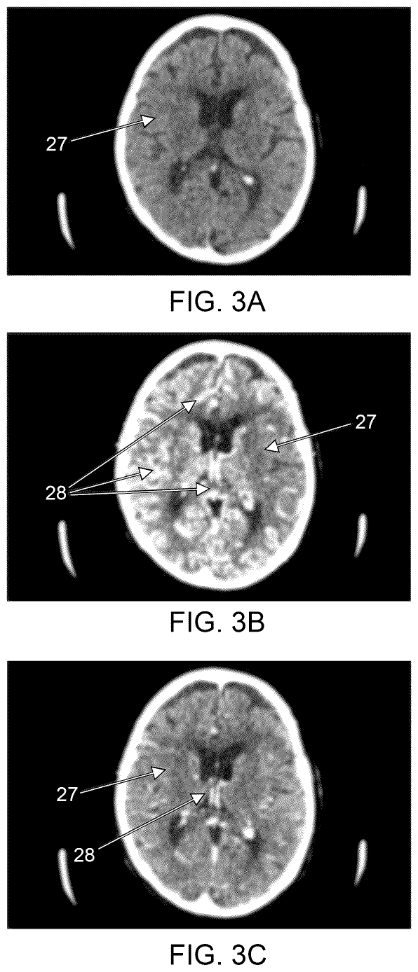

FIGS. 3A to 3C illustrate images representing an anatomical region of a patient, which have been acquired at different points in time;

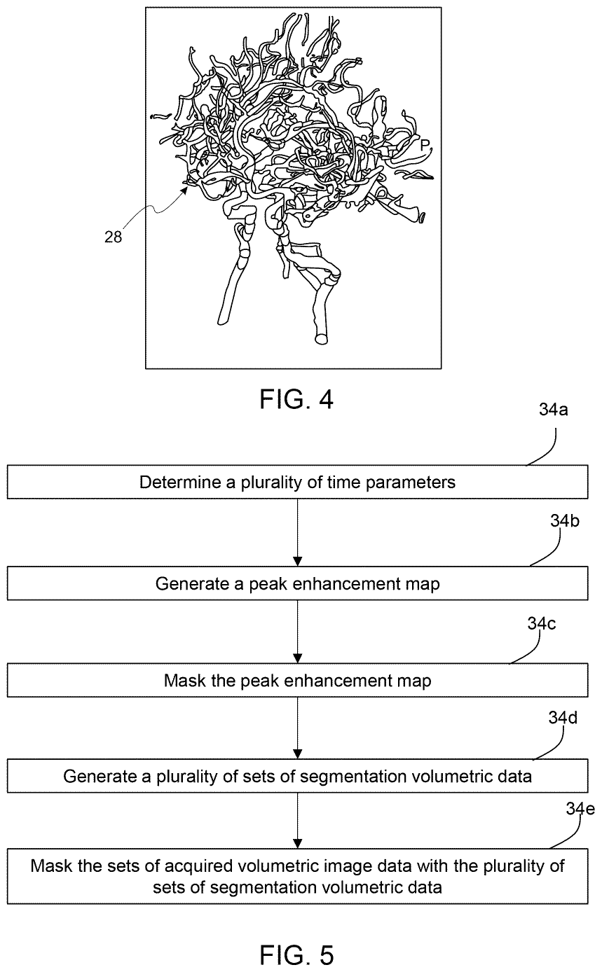

FIG. 4 illustrates an image representing a segmentation of an exemplary region of interest;

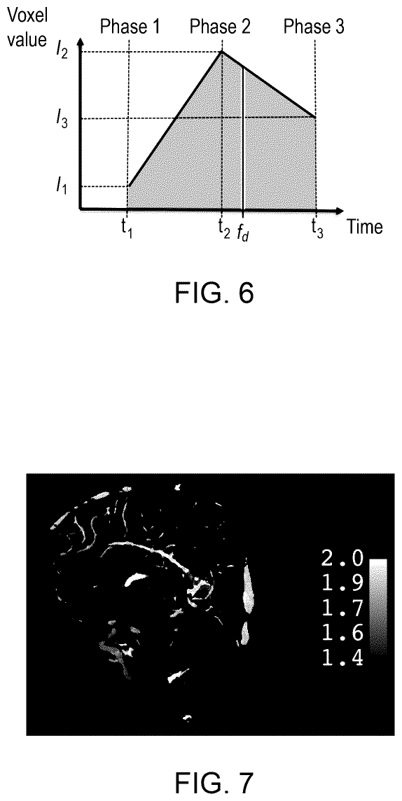

FIG. 5 is a flow chart illustrating in overview further stages of the process of FIG. 2;

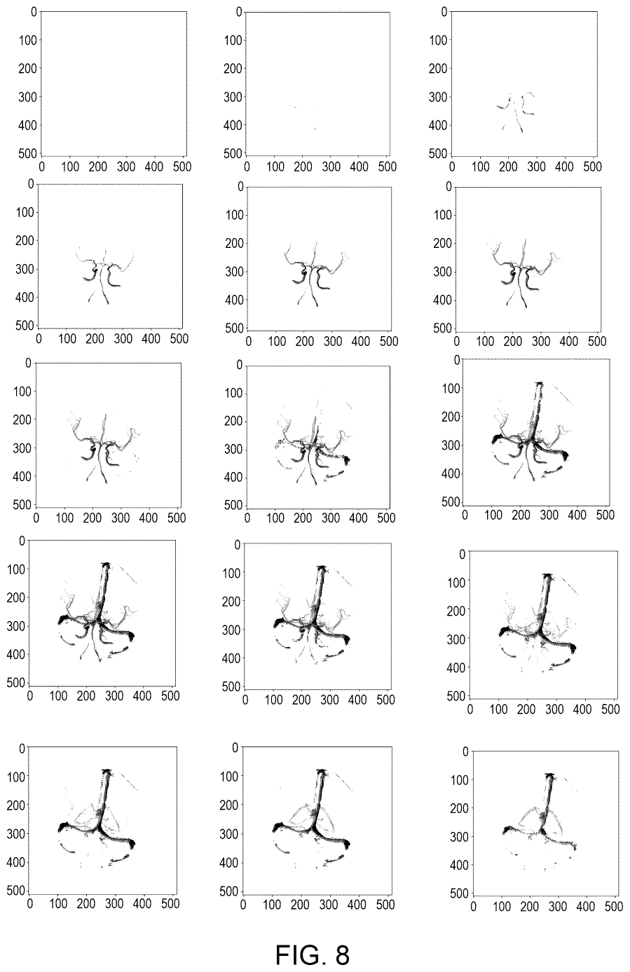

FIG. 6 illustrates an exemplary graph of a value of corresponding voxels over time;

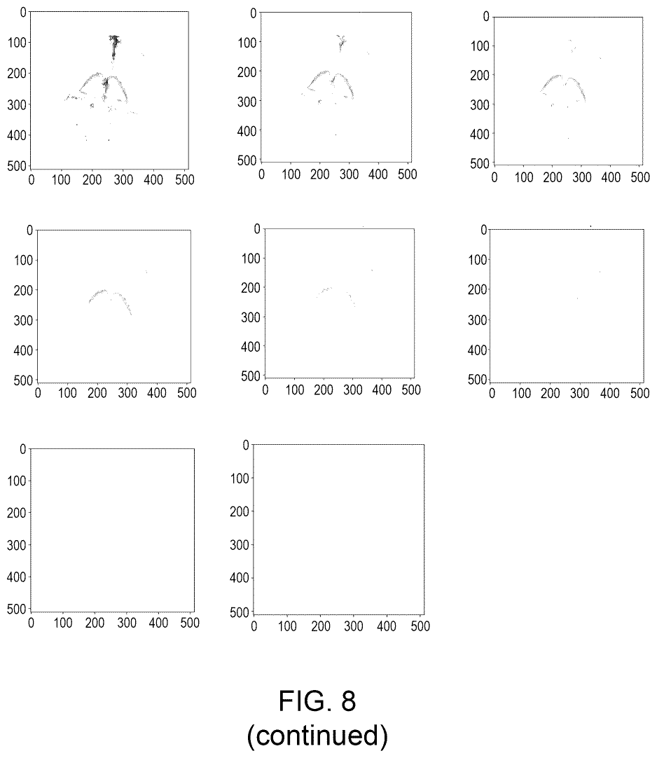

FIG. 7 illustrates a two-dimensional (2D) image representing a slice through an exemplary fractional peak enhancement that was generated using the further stages of FIG. 5;

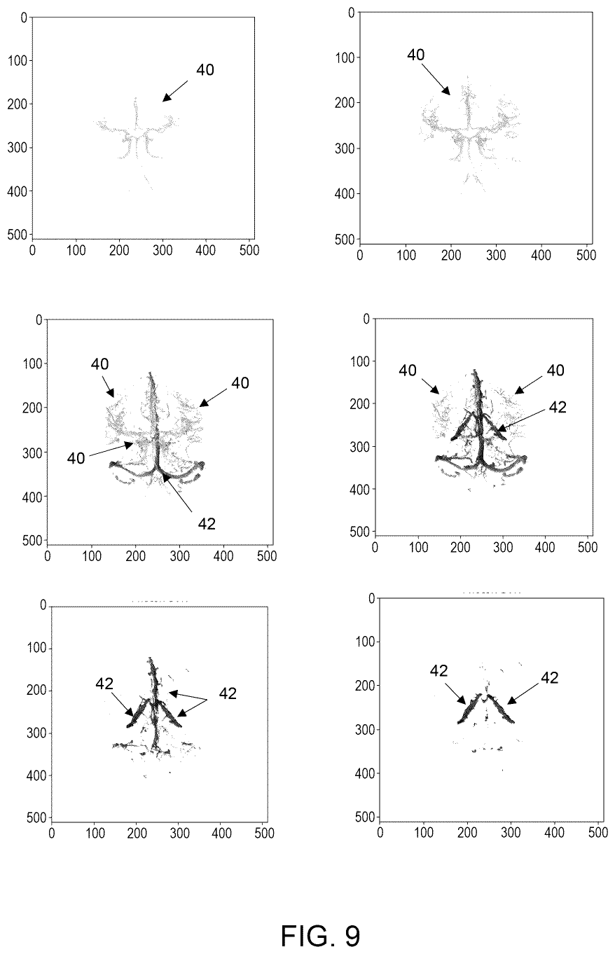

FIG. 8 illustrates an exemplary image sequence that has been generated using the process of FIG. 2;

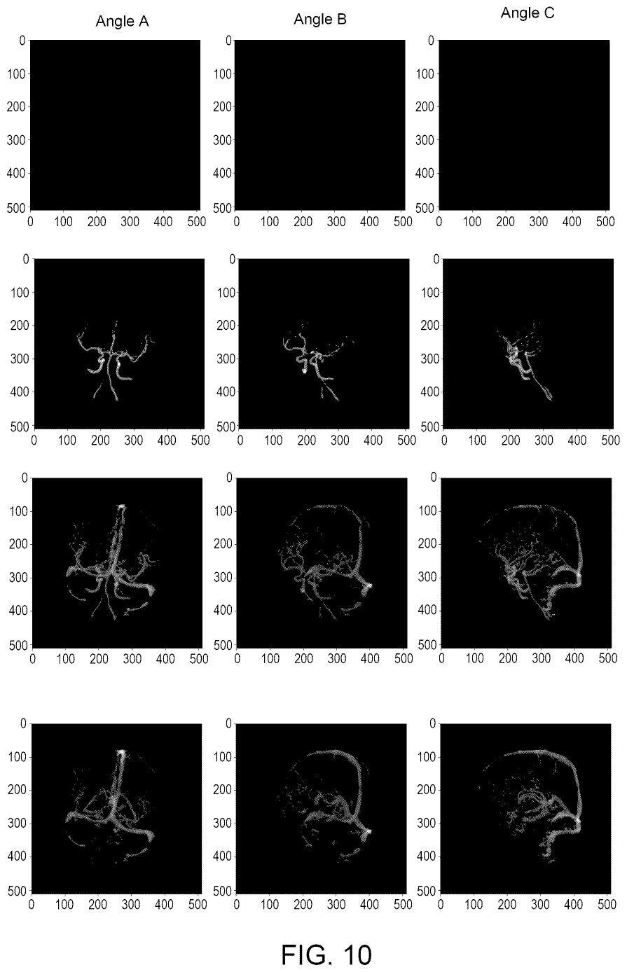

FIG. 9 illustrates another exemplary image sequence that has been generated using the process of FIG. 2;

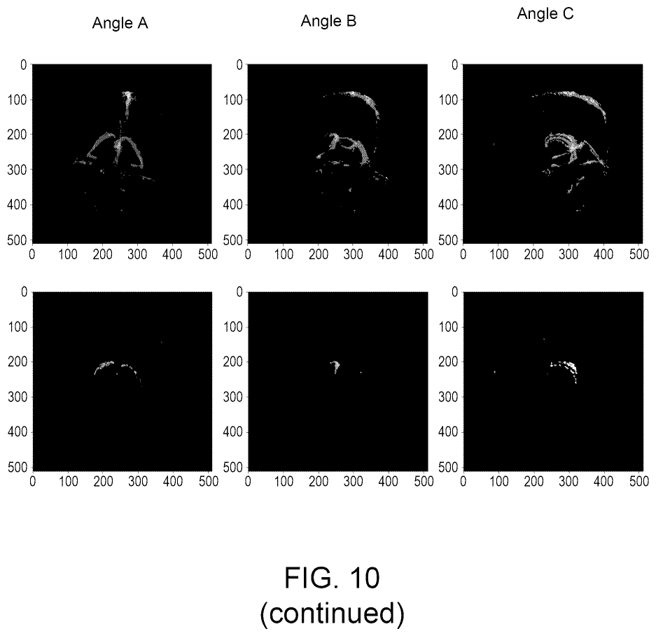

FIG. 10 illustrates another exemplary image sequence that has been generated using the process of FIG. 2;

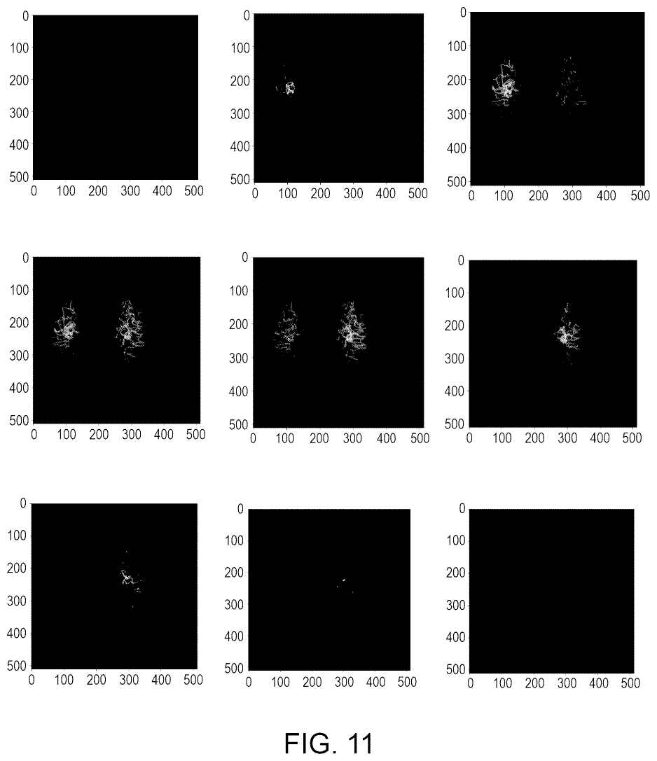

FIG. 11 illustrates another exemplary image sequence that has been generated using the process of FIG. 2; and

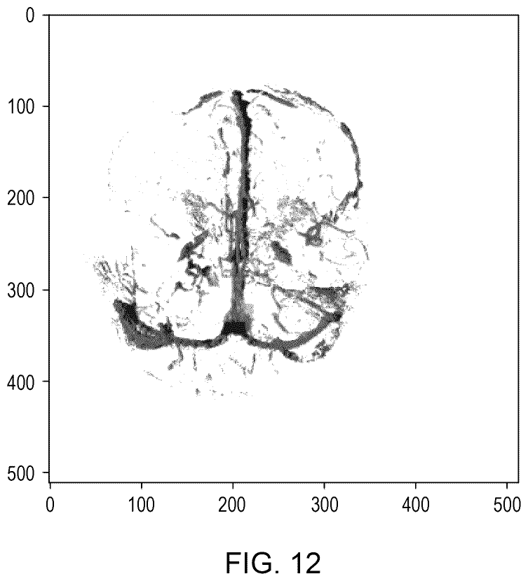

FIG. 12 illustrates an image representing a temporal average of an image sequence that has been generated using the process of FIG. 2.

Certain embodiments provide a medical image processing apparatus comprising processing circuitry configured to acquire a plurality of sets of volumetric image data of an anatomical region of a patient or other subject, each set of acquired volumetric image data corresponding to a respective different first point in time, generate a segmentation of a region of interest of the anatomical region, generate a plurality of sets of intermediate volumetric image data, one or more of the plurality of sets of intermediate volumetric image data representing the region of interest at a different second point in time, each second point in time corresponding to a point in time between two or more first points in time, and generate an image sequence based on the plurality of sets of intermediate volumetric image data, the image sequence representing at least a part or all of the region of interest.

The processing circuitry may be configured to generate the plurality of sets of intermediate volumetric image data based on voxel values of the plurality of sets of acquired volumetric image data.

At least one of the plurality of sets of intermediate volumetric image data may represent the region of interest at a first point in time.

The processing circuitry may be configured to generate an average of the plurality of sets of acquired volumetric image data. The processing circuitry may be configured to generate the segmentation of the region of interest of the anatomical region based on the average. Alternatively, the processing circuitry may be configured to generate the segmentation of the region of interest based on each of the plurality of sets of acquired volumetric image data.

The processing circuitry may be configured to determine a plurality of time parameters. Each time parameter may be determined based on voxel values of corresponding voxels of the plurality of sets of acquired volumetric image data. Each time parameter of the plurality of time parameters may represent a point in time at which a value of the corresponding voxels of the plurality of sets of volumetric image data is likely to be maximum. The processing circuitry may be configured to generate peak enhancement volumetric data based on the plurality of time parameters.

The peak enhancement volumetric data may comprises a plurality of voxels. A value of each of the plurality of voxels may be represented by a respective time parameter.

The processing circuitry may be configured to determine each time parameter (f_{d}) according to

$${f}_{d} = \frac{{Σ}_{p} {I}_{p} {t}_{p}}{{Σ}_{p} {I}_{p}} ,$$

wherein Iₚ corresponds to a voxel value at a respective first point in time tₚ.

The processing circuitry may be configured to determine each time parameter (f_{d}) according to

$${f}_{d} = \frac{{Σ}_{p} {I}_{p}^{n} {t}_{p}}{{Σ}_{p} {I}_{p}^{n}} ,$$

wherein Iₚ corresponds to a voxel value at a respective first point in time tₚ, and n is an exponent.

The processing circuitry is configured to mask the peak enhancement volumetric data using the segmentation of the region of interest.

The processing circuitry may be configured to determine a threshold based on the peak enhancement volumetric data. The processing circuitry may be configured to apply the threshold to the peak enhancement volumetric data. The processing circuitry may be configured to vary a value of the threshold. The processing circuitry may be configured to generate a set of segmentation volumetric data for each value of the threshold. Alternatively, the processing circuitry may be configured to apply a machine learning algorithm to the peak enhancement volumetric data to generate a plurality of sets of segmentation volumetric data.

The processing circuitry may be configured to aggregate the plurality of sets of acquired volumetric image data to a single set of acquired volumetric image data. The processing circuitry may be configured to mask the single set of acquired volumetric image data with at least one or each set of segmentation volumetric data. Alternatively, the processing circuitry may be configured to mask the average of the plurality of sets of acquired volumetric image data with at least one or each set of segmentation volumetric data to generate the plurality of sets of intermediate volumetric image data.

The processing circuitry may be configured to render each of the plurality of sets of intermediate volumetric image data, e.g. to generate a plurality of images representing at least the part or all of the region of interest. One or more of the plurality of images may correspond to a respective different second point in time. At least one of the plurality of images may correspond to a first point in time.

The image sequence may comprise the plurality of images.

The processing circuitry may be configured to indicate an image in the image sequence as representing at least the part or all of the region of interest at a first point in time.

The region of interest may comprise a first part and a second part. The processing circuitry may be configured to render volumetric image data of each set of intermediate volumetric image data associated with the first part separately from volumetric image data of each set of intermediate volumetric image data associated with the second part.

The processing circuitry may be configured to only render volumetric image data of each set of intermediate volumetric image data associated with a part of the region of interest.

The processing circuitry may be configured to render each set of intermediate volumetric image data such that different parts of the region of interest are represented by different colours associated with different points in time.

The region of interest may comprise an anatomical structure or a part thereof. The region of interest may comprise a region that surrounds the anatomical structure or a part thereof. The region of interest may comprise a region or other anatomical structure adjacent to the anatomical structure or part thereof.

The anatomical region may comprise a brain of the patient or other subject. The region of interest may comprise at least a part or all of a vasculature of the brain.

The anatomical region may comprise a liver of the patient or other subject. The region of interest may comprise one or more vessels of the liver.

Certain embodiments provide a medical image processing method comprising acquiring a plurality of sets of volumetric image data of an anatomical region of a patient or other subject, each set of acquired volumetric image data corresponding to a respective different first point in time, generating a segmentation of a region of interest of the anatomical region, generating a plurality of sets of intermediate volumetric image data, one or more of the plurality of sets of intermediate volumetric image data representing the region of interest at a different second point in time, each second point in time corresponding to a point in time between two or more first points in time, and generating an image sequence based on the plurality of sets of intermediate volumetric image data, the image sequence representing at least a part or all of the region of interest.

A medical image processing apparatus 10 according to an embodiment is schematically illustrated in FIG. 1. The medical image processing apparatus 10 comprises a computing apparatus 12, which may be provided in the form of a personal computer (PC) or workstation. In this embodiment, the computing apparatus 12 is connected to a scanner 14, e.g. via a data store 16. However, it will be appreciated that in other embodiments, the medical image processing apparatus may not be connected or coupled to any scanner.

The medical image processing apparatus 10 further comprises one or more display screens 18 and an input device or devices 20, such as a computer keyboard, mouse or trackball.

In the present embodiment, the scanner 14 is a computed tomography (CT) scanner. However, it will be appreciated that in other embodiments the scanner may comprise another medical scanner, such as a nuclear magnetic resonance scanner, an ultrasound scanner or another medical scanner. The scanner 14 is configured to generate volumetric image data representing an anatomical region of a patient or other subject.

Volumetric image data comprises a plurality of voxels arranged in a three-dimensional (3D) grid. Each voxel has a voxel value associated with it. The voxel values represent measurements of a physical parameter. For example, in the case of CT scans, the voxel values represent the opacity of those voxels to X-rays, i.e. their X-ray stopping power. X-ray stopping power is measured in Hounsfield units (HUs) which is closely correlated with density (mass per unit volume).

In the present embodiment, volumetric image data sets obtained by the scanner 14 are stored in the data store 16 and subsequently provided to the computing apparatus 12. In an alternative embodiment, volumetric image data sets may be supplied from a remote data store (not shown). The data store 16 or remote data store may comprise any suitable form of memory storage.

The computing apparatus 12 comprises a processing circuitry 22 for processing of data. The processing circuitry 22 comprises a central processing unit (CPU) and Graphical Processing Unit (GPU). The processing circuitry 22 provides a processing resource for automatically or semi-automatically processing volumetric image data sets and/or medical image data set. In other embodiments, the data to be processed may comprise any image data, which may not be medical image data.

In the present embodiment, the computing apparatus 12 comprises rendering circuitry 24 configured to generate a two-dimensional (2D) image from volumetric image data representing the anatomical region or a part thereof. The rendering circuitry 24 is part of the processing circuitry 22. The processing circuitry 22 comprises display circuitry 26 configured to display the 2D images or an image sequence, which comprises the 2D image, to a user on the display screen 18. It will be appreciated that in other embodiments, the rendering circuitry and/or the display circuitry may be provided separately from the processing circuitry.

In the present embodiment, the circuitries 22, 24, 26 are each implemented in the CPU and/or GPU by means of a computer program having computer-readable instructions that are executable to perform one or more operations of the medical image processing apparatus 10 and/or a medical image processing method of an embodiment described herein. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for the sake of clarity.

FIG. 2 is a flow chart illustrating in overview a process of an embodiment.

At stage 30, the processing circuitry 22 is configured to acquire a plurality of sets of volumetric image data of an anatomical region of a patient or other subject. For example, the processing circuitry 22 may be configured to acquire the plurality of sets of volumetric image data from the data store 16. Volumetric image data may also be referred to as volumetric imaging data. The terms “volumetric image data” and volumetric imaging data” may be interchangeably used.

The plurality of sets of volumetric image data comprise multi-phase CT image data. Each set of volumetric image data corresponds to a respective different point in time. The points in time will be referred to in the following as first points in time. The first points in time may be considered as encompassing the term acquisition points in time. For example, each set of volumetric image data corresponds to a phase of a multi-phase CT scan, such as a contrast-enhanced multi-phase CT scan. Each phase of the multi-scan CT corresponds to a respective first point in time. The scanner 14 may be configured to perform a plurality of CT scans after injection of a contrast material into the anatomical region to generate the multi-phase CT image data.

The multi-phase CT scan may generate time-resolved volumetric image data, such as time-resolved perfusion volumetric image data, of the anatomical region of the patient or other subject. The multi-phase CT scan may also be referred to as multi-phase CT angiography or CT angiography (CTA).

In the present embodiment, the plurality of sets of acquired volumetric image data comprises three sets of acquired volumetric image data of the anatomical region of the patient or other subject. However, it will be appreciated that in other embodiments, the plurality of sets of acquired volumetric image data may comprise more or less than three sets of acquired volumetric image data of the anatomical region of the patient or other subject. For example, a number of the sets of acquired volumetric image data may be such that each set of volumetric image data corresponds to a discrete set of volumetric image data. Additionally or alternatively, the number of sets of acquired volumetric image data may be less than a number of sets of volumetric image data that are acquired in a four-dimensional (4D) CT scan and/or more than one. For example, the number of sets acquired in 4D CT scans may be about twenty. However, it will be appreciated that the number of sets acquired in 4D scans may be more or less than twenty. The sets of acquired volumetric image data may be referred to sets of original volumetric image data or original CT values.

FIGS. 3A to 3C show images representing an anatomical region of a patient or other subject. The images shown in FIGS. 3A to 3C have been acquired using a multi-phase CT scan. Each of the images shown in FIGS. 3A to 3C represents the anatomical region at a respective different first point in time. In this embodiment, the anatomical region 27 comprises the brain of the patient or other subject.

In some embodiments, only a region of the anatomical region 27 may be of interest. This region will be referred to in the following as the region of interest, which is indicated in FIGS. 3A to 3C by reference numeral 28. The region of interest 28 of the anatomical region of the patient or other subject may comprise an anatomical structure or organ. The anatomical structure may comprise an organ, one or more blood vessels or any other anatomical structure, or a combination of anatomical structures. A contrast material may be injected into the anatomical structure. In the present embodiment, the anatomical structure comprises the vasculature, e.g. one or more blood vessels, of the brain of the patient or other subject.

The image shown in FIG. 3A has been acquired at a first point in time, when no contrast material was present in the anatomical region. This may also be referred to as a first phase of the multi-phase CT scan.

The image shown in FIG. 3B has been acquired at another first point in time. For example, the image shown in FIG. 3B has been acquired subsequent to the image shown in FIG. 3A. In the image shown in FIG. 3B, a plurality of blood vessels are visible. This is due to the image shown in FIG. 3B having been acquired at a first point in time, when most of the contrast material was present in the anatomical region 27 of the patient or other subject. This may also be referred to as a second phase of the multi-phase CT scan.

The image shown in FIG. 3C has been acquired at another first point in time. For example, the image shown in FIG. 3C has been acquired subsequent to the image shown in FIG. 3B. In the image shown in FIG. 3C, a number of the visible blood vessels has decreased relative to the number of visible blood vessels in the image shown in FIG. 3B. This is due to the image shown in FIG. 3C having been acquired at a first point in time, when contrast material in the anatomical region 27 is mostly depleted. This may also be referred to as a third phase of the multi-phase CT scan.

It can be seen from FIGS. 3A to 3C that a level of contrast material in the anatomical structure varies between the different first points in time. In the embodiment shown in FIGS. 3A to 3C, only three CT scans were acquired, which may make it difficult to assess the flow in blood vessels and/or filling of blood vessels.

It will be appreciated that in other embodiments, the anatomical region may comprise a different part or a different organ of the patient or other subject. For example, in other embodiments, the anatomical region may be comprise the liver of the patient or other subject. In such other embodiments, the region of interest may comprise a vasculature, e.g. one or more vessels, of the liver.

At stage 32, the processing circuitry 22 is configured to generate a segmentation of a region of interest of the anatomical region.

In the present embodiment, the processing circuitry 22 is configured to generate an average of the plurality of sets of acquired volumetric image data. The average comprises a temporal average of the plurality of sets of acquired volumetric image data. For example, each voxel of the temporal average of the sets of acquired volumetric image data may comprise an average of the voxel values (e.g. HU values) at the first points in time.

In some embodiments, the temporal average may comprise a weighted temporal average of the plurality of sets of acquired volumetric image data. Alternatively, the temporal average comprises a weighted temporal variance. An exemplary method, which may be used for generating the weighted temporal average or the weighted temporal variance of the plurality sets of acquired volumetric image data, is described in Meijs et al. “Robust Segmentation of the Full Cerebral Vasculature in 4D CT of Suspected Stroke Patients”, Scientific Reports 7, 15622 (2017). It will be appreciated that any other suitable method may be used for generating the weighted temporal average or weighted temporal variance of the plurality sets of acquired volumetric image data. The weighting may be used to adjust for changes in a radiation dose used in the CT scans. By using a weighting to generate the temporal average or temporal variance, a signal to noise ratio may be improved. However, it will be appreciated that in other embodiments, the plurality sets of acquired volumetric image data may be averaged without a weighting.

In the present embodiment, the processing circuitry 22 is configured to generate the segmentation of the region of interest based on the temporal average. For example, the processing circuitry 22 may segment the region of interest in the temporal average.

In other embodiments, the processing circuitry may be configured to generate the segmentation of the region of interest based on each of the plurality of sets of acquired volumetric image data. This may generate a plurality of sets of segmented volumetric image data. For example, the processing circuitry may be configured to segment the region of interest in each set of acquired volumetric image data separately. In this embodiment, the segmentation of the region of interest may comprise an average, e.g. a temporal average or temporal variance, of the plurality of sets of segmented volumetric image data. In such embodiments, the average may be a weighted temporal average or weighted temporal variance. For example, the processing circuitry 22 may be configured to apply an OR function or any other suitable function to the plurality of sets of segmented volumetric image data to generate the average. However, it will be appreciated that in other embodiments, the processing circuitry may use a different method or process for generating the average of the plurality of sets of segmented volumetric image data.

The processing circuitry 22 is configured to use a thresholding process, machine learning algorithm, such as a convolutional neural network algorithm, and/or any other suitable segmentation process or algorithm to generate the segmentation of the region of interest of the anatomical region of the patient or other subject.

The processing circuitry 22 is configured to use the segmentation of the region of interest as a mask, as will be described below in more detail.

FIG. 4 shows an image representing an exemplary segmentation of a region of interest of an anatomical region. As described above, in this embodiment, the region of interest 28 comprises the vasculature of the brain of the patient or other subject.

In some embodiments, the region of interest comprises the anatomical structure and a region that surrounds the anatomical structure. Alternatively or additionally, the region of interest comprises a region or other anatomical structure adjacent to the anatomical structure. For example, in some embodiments, the anatomical structure comprises the vasculature of the brain and surrounding and/or adjacent tissue of the brain that may be perfused. In such embodiments, a segmentation mask, which may be used to generate the segmentation of the region of interest, may be dilated to include the surrounding and/or adjacent region.

In some embodiments, the region of interest comprises a part of the anatomical structure, a region surrounding and/or adjacent to the part of the anatomical structure. For example, in embodiments where the anatomical structure comprises the vasculature of the brain, the part of region of interest may comprise the middle cerebral artery. The region of interest may additionally comprise a region surrounding and/or adjacent to the middle cerebral artery. In such examples, the region of interest may be referred to as the middle cerebral artery (MCA) vascular region. However, it will be appreciated that in other embodiments, the region of interest may comprise another part of the anatomical structure or another anatomical structure.

At stage 34, the processing circuitry 22 is configured to generate a plurality of sets of intermediate volumetric image data. One or more of the plurality of sets of intermediate volumetric image data represents the region of interest at a different second point in time. Each second point in time may also be referred to as an intermediate point of time. Each second point in time corresponds to a point in time between two or more first points in time. In some embodiments, at least one of the plurality of sets of intermediate volumetric image data represents the region of interest at a first point in time.

Stage 34 will be described in more detail below.

At stage 36, the processing circuitry 22 is configured to generate an image sequence based on the plurality of sets of intermediate volumetric image data. The image sequence represents at least a part or all of the region of interest. The image sequence comprises a plurality of images. Each image represents at least the part or all of the region of interest at a respective different point in time. For example, each image corresponds to a frame of the image sequence. The image sequence may also be referred to as a 2D video sequence.

In the present embodiment, the processing circuitry 22 is configured to render each set of intermediate volumetric image data to generate a respective image. The image represents the region of interest at a respective second point in time. In some embodiments, the image may alternatively represent the region of interest at a respective first point in time. The images are part of the image sequence mentioned above.

In this embodiment, the rendering circuitry 24 is configured to render each set of intermediate volumetric image data. For example, the rendering circuitry 24 is configured to use a projection or rendering algorithm to generate each image. The projection algorithm may comprise an Average Intensity Projection (AVIP) algorithm, a Maximum Intensity Projection (MIP) algorithm, a global rendering algorithm or any other suitable projection or rendering algorithm.

At stage 38, the processing circuitry 22 is configured to display the image sequence. For example, the display circuitry 26 is configured to display the image sequence on the display screen 18 to a user.

FIG. 5 is a flow chart illustrating in overview further stages of the process shown in FIG. 2. One or more of the stages shown in FIG. 5 may be part of stage 34 shown in FIG. 2.

At stage 34a, the processing circuitry 22 is configured to determine a plurality of time parameters. The processing circuitry 22 is configured to determine each time parameter based on the plurality of sets of acquired volumetric image data. For example, the processing circuitry 22 is configured to determine each time parameter based on voxel values of corresponding voxels of the plurality of sets of acquired volumetric image data. The corresponding voxels of the plurality of sets of volumetric image data may be considered as voxels sharing a same voxel position, e.g. in the 3D grid, in the plurality of sets of acquired volumetric image data. For example, the processing circuitry 22 may be configured to determine a time parameter for each voxel position of corresponding voxels of the plurality of sets of acquired volumetric image data. The time parameter represents a point in time, e.g. a first point in time or a second point in time, at which a value of the corresponding voxels is likely to be maximum or enhanced. For example, the time parameter represents a centre or peak of the value of the corresponding voxels.

The processing circuitry 22 is configured to determine each time parameter (f_{d}) according to:

$${f}_{d} = \frac{{\sum }_{p} {I}_{p} {t}_{p}}{{\sum }_{p} {I}_{p}} \qquad ( Equation 1 )$$

wherein Iₚ corresponds to a voxel value at a respective first point in time tₚ. For example, Iₚ corresponds to a voxel value of a p^{th} voxel at a p^{th} first point in time tₚ.

In some embodiments, the processing circuitry 22 is configured to determine each time parameter (f_{d}) according to:

$${f}_{d} = \frac{{\sum }_{p} {I}_{p}^{n} {t}_{p}}{{\sum }_{p} {I}_{p}^{n}} \qquad ( Equation 2 )$$

wherein Iₚ corresponds to a voxel value at a respective first point in time tₚ, and n is an exponent. For example, Iₚ corresponds to a voxel value of a p^{t}h voxel at a p^{t}h first point in time tₚ. The exponent may be used to accentuate differences between two or more corresponding voxel values Iₚ at respective first points in time tₚ. In some embodiments, n equals 2. However, it will be appreciated that in other embodiments, the exponent may be less or more than 2. The time parameter may also be referred to as a fractional delay or relative filing delay.

FIG. 6 shows an exemplary graph of a value of corresponding voxels overtime. In the example shown in FIG. 6, p equals 3. The first points in time are labelled as t₁, t₂ and t₃ and the associated voxel values at these first points in time are labelled as I₁, I₂ and I₃ in FIG. 6. At t₁, a first CT scan has been performed by the scanner 14, which results in a first set of acquired volumetric image data. This is referred to as Phase 1 in FIG. 6. At t₂, a second CT scan has been performed by the scanner 14, which results in a second set of acquired volumetric image data. This is referred to as Phase 2 in FIG. 6. At t₃, a third CT scan has been performed by the scanner 14, which results in a third set of acquired volumetric image data. This is referred to as Phase 3 in FIG. 6. In the example shown in FIG. 6, a maximum of the value of the corresponding voxels lies at t₂. However, a relationship between the voxel value at t₂ and the voxel value at t₃ differs from a relationship between the voxel value at t₁ and the voxel value at t₃. Based on the voxel values I₁, I₂ and I₃ and the first points in time t₁, t₂ and t₃, the processing circuitry 22 is configured to determine the time parameter f_{d}, e.g. according to Equation 1 or Equation 2. It can be seen in FIG. 6 that the determined time parameter f_{d} lies at a point in time between the first points in time t₂ and t₃ and proximal to the first point in time t₂. A maximum of the voxel value of the corresponding voxels is expected between the first points in time t₂ and t₃ and not at t₂. As such, a likely distribution of the value of the corresponding voxels in dependence on time may differ from that shown in FIG. 6. The terms “voxel value” or “value of a/the voxel” may be interchangeably used with the term “intensity.” It will be appreciated that the time parameter is not limited to lying between the between the first points in time t₂ and t₃ and that in other examples the time parameter may lie at a different time. For example, where the voxel value I₃ is low and equal to I₁, the time parameter f_{d} may be equal to t₂. In examples, where no time parameters are generated and only the sets of acquired volumetric image data are used to generate intermediate sets of volumetric image data, the first points in time t₁, t₂ or t₃ may be considered as the points in time at which a voxel value of the corresponding voxels is likely to be the highest. The determination of the time parameter, as described above, may improve an estimate of a point in time at which the voxel value of corresponding voxels is likely to be maximum compared to only considering the voxel values at the first points in time. The determination of the time parameter may provide information to predict a fractional peak time, e.g. a point in time at which the voxel value of the corresponding voxels is likely to be the highest.

At stage 34b, the processing circuitry 22 is configured to generate a peak enhancement volumetric data based on the plurality of time parameters. The peak enhancement volumetric data may take the form of a peak enhancement map, such as a fractional peak enhancement map (FPEM). The fractional peak enhancement map comprises a plurality of voxels. A value of each voxel of the peak enhancement map is represented by a respective time parameter. As described above, the time parameter is determined for each voxel position of corresponding voxels of the acquired set of volumetric image data at the first points in time, e.g. according Equation (1) or (2). The fractional peak enhancement map may also be referred to as a filing delay map, a HU weighted map, a weighted temporal average map, a delay map or a fractional delay map.

FIG. 7 shows a two-dimensional (2D) image representing a slice through an exemplary fractional peak enhancement map. The fractional peak enhancement map was generated using the process described in relation to FIGS. 2 and 6. A slice of volumetric image data may be formed by performing a multi-planar reformatting (MP R) technique or any other suitable technique.

The first points in time t₁, t₂ and t₃ are each associated with a value. The value may represent an intensity value. The value may be a floating point value. The value may be predetermined. In this embodiment, the value represents an intensity of the fractional peak enhancement map. The value increases with increasing time. For example, the first point in time t₁ is associated with the lowest value. The lowest value represents by the lowest intensity in the fractional peak enhancement map. In this example, the lowest value is 0. Each subsequent first point in time is associated with an increased value relative to a preceding first point in time. For example, the first point in time t₂ is associated with a value of 1 and the first point in time t₃ is associated with a value of 2. The highest value represent the highest intensity in the fractional peak enhancement map. In the fractional peak enhancement map, a higher intensity, e.g. higher values, is associated with parts of the anatomical structure, e.g. veins, that are filled with the contrast material at a later point in time. A lower intensity, e.g. lower values, is associated with parts, e.g. arteries or proximal arteries, of the anatomical structure that are filled with the contrast material first. E ach point in time represented by a time parameter in the fractional peak enhancement map is associated with a respective value. For example, each point in time represented by a time parameter in the fractional peak enhancement map is associated with a value of one of: the first points in time t₁, t₂ or t₃ or a value between at least two values associated with at least two of: the first points in time t₁, t₂ and t₃. For example, each point in time represented by the time parameter may be associated with a value between 0 and 2. As such, each voxel of the fractional peak enhancement map may be also associated with an intensity. It will be appreciated that the process described herein is not limited to the exemplary values described above and/or that in other embodiments, one or more of the first points in time and the time parameters may be associated with different values. In other embodiments, the value may represent a brightness value.

In some embodiments, the processing circuitry 22 is configured to filter the fractional peak enhancement map. For example, the processing circuitry 22 is configured to use a smoothing filtering method, such as an anisotropic diffusion filtering method or any other suitable smoothing method, to filter the fractional peak enhancement map.

At stage 34c, the processing circuitry 22 is configured to mask the fractional peak enhancement map using the segmentation of the region of interest, which was generated at stage 32 shown in FIG. 2. The processing circuitry 22 is configured to mask the fractional peak enhancement map with the segmentation of the region of interest, e.g. such that the masked fractional peak enhancement map represents the region of interest of the anatomical region only. For example, any values of voxels that are not part of the region of interest may be set to zero.

At stage 34d, the processing circuitry 22 is configured to generate a plurality of sets of segmentation volumetric data. One or more sets of segmentation volumetric data comprises volumetric image data representing the region of interest at a different second point in time. At least one set of segmentation volumetric data may comprise volumetric image data representing the region of interest at a first point in time. A set of segmentation volumetric data may also be referred to as a mask, a segmentation or a segmentation mask.

The processing circuitry 22 is configured to generate the plurality of sets of segmentation volumetric data based on the masked fractional peak enhancement map. A number of the sets of segmentation volumetric data may determine a number of frames of the image sequence. The number of the sets of segmentation volumetric data may be selected based on a desired or given temporal resolution of the image sequence. The sets of segmentation volumetric data may also be referred to as M volumes, M vessel segmentations or M intermediate vessel segmentations.

In the present embodiment, the processing circuitry 22 is configured to apply a thresholding method or process to the masked fractional peak enhancement map. For example, the processing circuitry 22 is configured to apply a threshold to the masked fractional peak enhancement map. The processing circuitry 22 is configured to determine the threshold based on the time parameter associated with each voxel of the fractional peak enhancement map. For example, the processing circuitry 22 may be configured to determine the threshold based on a distribution of the time parameter associated with each voxel of the fractional peak enhancement map. The threshold may correspond to a value associated with a point in time represented by the time parameter. For example, the threshold may be between 0 and 2. However, it will be appreciated that in other embodiments, a different threshold may be selected.

The processing circuitry 22 is configured to vary a value of the threshold. The processing circuitry 22 is configured to generate a set of segmentation volumetric data for each value of the threshold. By varying the value of the threshold, the sets of segmentation volumetric data may be generated to represent the region of interest at different points in time. For example, the threshold value may be such that one or more generated sets of segmentation volumetric data are associated with one or more time parameters having a low value. This may allow the filling of proximal arteries of the vasculature with contrast material to be initially visualised. The threshold value may be increased such that one or more generated sets of segmentation volumetric data are associated with one or more time parameters having an intermediate value. This may allow the filling of more distal arteries of the vasculature with contrast material to be visualised. The threshold value may be increased such that one or more generated sets of segmentation volumetric data are associated with one or more time parameters having a high value. This may allow the filling of distal arteries and veins of the vasculature with contrast material to be visualised.

In some embodiments, each threshold comprises a one-sided threshold. The processing circuitry 22 may be configured to increase the value of the threshold. In embodiments where the region of interest comprises the vasculature of the brain of the patient or other subject, the threshold may be selected such that the respective segmentation represents parts, e.g. blood vessels, of the vasculature that are filled with contrast material early. With increasing threshold, the corresponding generated sets of segmentation volumetric data represent further parts, e.g. further blood vessels, of the vasculature. For example, the one-sided threshold may be used to illustrate the continuous filling of the vasculature with contrast material. In this example, the blood vessels may be illustrated as remaining filled.

In other embodiments, each threshold comprises a two-sided threshold. For example, the threshold comprises a first bound and a second bound. The first and second bounds of the threshold may define a window of time parameter values. The first and second bounds of the threshold may be varied, e.g. so that a sliding window is passed over the masked fractional peak enhancement map. This may also be referred to as windowing of the masked fractional peak enhancement map. A width of the window may define how much time a set of segmentation volumetric data represents.

For example, a value of the first side or bound of the threshold may be increased and a value of the second side or bound may be decreased. The value of the first bound and the value of the second bound may be simultaneously varied. For example, the processing circuitry 22 may be configured to generate a first set of segmentation volumetric data using a value of 0 for the first bound of the threshold and a value of 0.2 for the second bound of the threshold. The processing circuitry 22 may be configured to generate a second set of segmentation volumetric data using a value of 0.1 for the first bound of the threshold and a value of 0.3 for the second bound of the threshold. The processing circuitry 22 may be configured to generate one or more additional sets of segmentation volumetric data using increased values for the first and second bounds. However, it will be appreciated that in other embodiments, only one of the first and second bounds may be varied or the first and second bounds may be varied at a different rate or time. The two-sided threshold may also be referred to as a double-sided threshold.

In embodiments where the region of interest comprises the vasculature of the brain of the patient or other subject, the threshold, e.g. the first and second bounds thereof, may be selected such that the respective set of segmentation volumetric data illustrates a flow of the contrast material in the vasculature. For example, the two-sided threshold may be used to illustrate parts, e.g. blood vessels, of the vasculature where the contrast material arrives early and then progressively parts, e.g. blood vessel, of the vasculature where the contrast material arrives late.

In other embodiments, the processing circuitry 22 is configured to apply a machine learning algorithm, such as convolutional neural network algorithm, to the masked fractional peak enhancement map to generate the plurality of sets of segmentation volumetric data. The machine learning algorithm may comprise a trained machine learning algorithm. For example, the machine learning algorithm may be trained using other volumetric image data acquired by the scanner 14 or another scanner or other medical image data, e.g. acquired using another imaging method, such as a fluoroscopy method or the like. In the present embodiments, the machine learning algorithm is trained using volumetric CT perfusion data and/or multi-phase CT image data sets. The use of the machine learning may aid in a reduction of noise in the sets of segmentation volumetric data.

In some embodiments, the processing circuitry 22 may be configured to set the threshold such that the generated set of segmentation volumetric data comprises volumetric image data representing the region of interest at a first point in time. For example, when the threshold comprises one of the values associated with the first points of time t₁, t₂ and t₃, the generated set of segmentation volumetric comprises volumetric image data representing the region of interest at the first point in time.

In some embodiments, the sets of segmentation volumetric data correspond to the sets of intermediate volumetric image data mentioned above. In such embodiments, the process proceeds to stage 36, which is shown in FIG. 2.

In some embodiments, the processing circuitry 22 may be configured to process the sets of segmentation volumetric data further. At stage 34e, the processing circuitry 22 is configured to mask the plurality of sets of acquired volumetric image data of the anatomical region with each of the plurality of sets of segmentation volumetric data. For example, the processing circuitry 22 is configured to mask the average of the plurality of sets of acquired volumetric image data with each set of segmentation volumetric data. Each set of segmentation volumetric data may represent a binary mask.

In other embodiments, the processing circuitry 22 is configured to aggregate the plurality of sets of acquired volumetric image data to a single set of acquired volumetric image data. For example, the processing circuitry 22 may be configured to use a projection algorithm, such as a Maximum Intensity Projection algorithm, to aggregate the plurality of sets of acquired volumetric image data to a single set of acquired volumetric image data. In such embodiments, the processing circuitry 22 is configured to mask the single set of acquired volumetric image data with each set of segmentation volumetric data. The single set of acquired volumetric image data may also be referred to as an aggregate of the plurality of sets of acquired volumetric image data.

By masking the acquired volumetric image data of the anatomical region with the sets of segmentation volumetric data, the sets of intermediate volumetric image data are generated. Each set of intermediate volumetric image data corresponds to the average or aggregate of the plurality of sets of acquired volumetric image data that has been masked with a different respective set of segmentation volumetric data. This may allow for the generation of sets of intermediate volumetric image data, which comprise voxel values that are closer to those present in the sets of acquired volumetric image data. As such, one or more images generated based on the sets of intermediate volumetric image data may look less artificial.

The processing circuitry 22 is configured to use a masking algorithm, such as an evolving masking algorithm or any other suitable masking algorithm. In other embodiments, the processing circuitry 22 is configured to multiply each of the sets of segmentation volumetric data with the average or aggregate of the plurality of sets of acquired volumetric image data, e.g. to generate each set of intermediate volumetric image data.

It will be appreciated that in some embodiments only some of stages of the process described in relation to FIGS. 2 and 5 may be performed and/or one or more stages of the process may differ. For example, in some embodiments, stages 34d and 34e may be omitted. In such embodiments, the processing circuitry 22, e.g. the rendering circuitry 24, may be configured to render the masked fractional peak enhancement map. In such embodiments, the processing circuitry 22, e.g. display circuitry 26, is configured to display a 2D image representing the masked fractional peak enhancement map.

It will further be appreciated that one or more stages of the process described in relation to FIGS. 2 and 5 may be performed in a different order.

In the above description, the processing circuitry 22 is configured to generate the plurality of sets of intermediate volumetric image data based on voxel values of the plurality of sets of acquired volumetric image data.

In other embodiments, the processing circuitry 22 is configured to generate the plurality of sets of intermediate volumetric image data based on a likely progression of contrast material in the region of interest and a size of the region of interest. In such embodiments, the region of interest comprises a vessel. However, it will be appreciated that the terms “region of interest” and “vessel” may be interchangeably used. The processing circuitry 22 is configured to determine at least one reference point in the vessel. The processing circuitry 22 is configured to determine a distance along the vessel from the reference point. The processing circuitry 22 is configured to detect one or more edges or endpoints, such as leading and/or trailing edges or endpoints, of the contrast material in the vessel, e.g. based on the plurality of sets of acquired volumetric image data. The processing circuitry 22 is configured to determine a likely distance of contrast material along the vessel based on the reference point in the vessel and the edges or endpoints for each first point in time. The processing circuitry 22 is configured to generate a distance map, e.g. a temporal distance map, based on the determined likely distances for each first time point. The processing circuitry 22 may be configured to apply a threshold to the distance map, as described in relation to stage 34d above, to generate the sets of intermediate volumetric image data.

FIG. 8 shows an exemplary image sequence that has been generated using the process described above in relation to FIGS. 2 to 7. The image sequence shown in FIG. 8 includes 23 images, which represent the region of interest at 23 different successive points in time after injection of the contrast into the region of interest. However, it will be appreciated that in other embodiments, the image sequence may comprise more or less than 23 images.

In this example, the region of interest comprises the vasculature of the brain of the patient or other subject. In this example, the rendering circuitry 24 has rendered the sets of intermediate volumetric image data and the sets of acquired volumetric image data using a shaded volume rendering (SVR) process, such as a global illumination (GI) process, to generate the images of the image sequence.

In this example, the sets of intermediate volumetric image data corresponding to the images of the image sequence were generated by applying a two-sided threshold to the masked fractional peak enhancement map, as described in relation to stage 34d.

The image sequence shown in FIG. 8 illustrates a temporal progression of the contrast material through the vasculature. For example, it can be seen from FIG. 7 that the contrast material flows through different parts of the vasculature of the brain at different points in time.

FIG. 9 shows another exemplary image sequence that has been generated using the process described above in relation to FIGS. 2 to 7. In this example, the processing circuitry 22, e.g. the rendering circuitry 24, is configured to render the sets of intermediate volumetric image data such that different parts of the region of interest are represented by different colours. The different colours are associated with different points in time. One or more vessels that are filled with the contrast material at one or more earlier points in time may be rendered in a colour that is different from one or more vessel that are filled with the contrast material at one or more later points in time. For example, the parts of the region of interest that are filled with the contrast material earlier may be represented in the fractional peak enhancement map by a low value of the time parameter. Parts of the region of interest that are filled with the contrast material later may be represented in the fractional peak enhancement map by a relatively higher value of the time parameter. The processing circuitry 22, e.g. the rendering circuitry 24, may be configured to render different voxels of the fractional peak enhancement, which are associated with different time parameters, with different colours. In FIG. 9, the vessels that are filled with the contrast material at the earlier points in time are indicated by the light colour and reference numeral 40. The vessels that are filled with the contrast material at the later points in time are indicated by the dark colour and reference numeral 42. It will be appreciated that in other embodiments, the sets of intermediate volumetric image data may be rendered with more or less than two colours.

The images shown in FIGS. 8 and 9 are generated based on the sets of intermediate volumetric image data.

In some embodiment, the processing circuitry 22 may be configured to indicate an image in the image sequence as representing the region of interest at a first point in time. For example, the processing circuitry 22 may be configured to indicate the image that represents the region of interest at the first point in time in the image sequence as a key frame. In such embodiments, the processing circuitry 22 is configured to mask each set of the acquired volumetric image data using the segmentation of the region of interest, which was generated at stage 32 shown in FIG. 2. The processing circuitry 22 may be configured to apply a threshold to each set of masked acquired volumetric image data, e.g. to generate an approximate subset mask. The processing circuitry 22 may be configured to compare the subset mask to the sets intermediate volumetric image data, e.g. using a comparison method, such as a Dice overlap method or another suitable comparison method. The processing circuitry 22 may be configured to compare the subset mask to the sets intermediate volumetric image data to identify a set of intermediate volumetric image data that is similar to subset mask. The processing circuitry 22 may be configured to indicate an image representing the identified set of intermediate volumetric image data as the key frame, e.g. as representing the region of interest at a first point in time.

FIG. 10 shows another exemplary image sequence that has been generated by the processing circuitry 22 using the process described in relation to FIGS. 2 to 7. The images shown in FIG. 10 have been generated for three different angles, which are labelled as “Angle A”, “Angle B” and “Angle C” in FIG. 10. The images below each of these labels have been generated for the same angle.

For each angle, 6 images represent the region of interest at 6 different successive points in time after injection of the contrast into the region of interest. In this example, the region of interest comprises the vasculature of the brain of the patient or other subject. In this example, the rendering circuitry 24 has rendered the sets of intermediate volumetric image data using a projection algorithm, such as an Average Intensity Projection (AVIP) algorithm. In this example, the sets of intermediate volumetric image data corresponding to the images of the image sequence were generated by applying a two-sided threshold to the masked fractional peak enhancement map, as described in relation to stage 34d.

FIG. 11 shows another exemplary image sequence that has been generated by the processing circuitry 22 using the process described in relation to FIGS. 2 to 7. The image sequence shown in FIG. 11 includes 9 images, which represent the region of interest at 9 different successive points in time after injection of the contrast material into the region of interest.

In the example shown in FIG. 11, the processing circuitry 22, e.g. the rendering circuitry 24, it configured to only render volumetric image data of the sets of intermediate volumetric image data associated with a part of the region of interest. In this example, the part of the region of interest comprises the middle cerebral artery (MCA) vascular region of the brain of the patient or other subject. The MCA vascular region may be considered as a region or territory of the brain that is fed or supplied by the MCA.

In the example shown in FIG. 11, the rendering circuitry 24 has rendered the sets of intermediate volumetric image data using a projection algorithm, such as a Maximum Intensity Projection (MIP) algorithm. In this example, the sets of intermediate volumetric image data corresponding to the images of the image sequence were generated by applying a two-sided threshold to the masked fractional peak enhancement map, as described in relation to stage 34d.

It will be appreciated that in other embodiments, the region of interest comprises a first part and a second part. For example, the first and second parts of the region of interest may each comprise a hemisphere of the brain of the patient or other subject. The processing circuitry 22, e.g. the rendering circuitry 24, is configured to render volumetric image data of the sets of intermediate volumetric image data associated with the first part separately from volumetric image data of the sets of intermediate volumetric image data associated with the second part. In examples, where each of the first and second parts of the region of interest comprise the hemisphere of the brain of the patient or other subject, the image sequence may represent a sagittal image sequence of at least one or each hemisphere separately. This may also be referred to as an image sequence representing a split-sagittal view.

It will be appreciated that in other embodiments, the image sequence may comprise more or less than the number of images shown in FIGS. 8 to 11.

FIG. 12 shows an image that has been generated using the process described in relation to FIGS. 2 to 7.

In this example, stages 34d and 34e were also omitted. Instead, at stage 64, the processing circuitry 22, e.g. the rendering circuitry 24, is configured to render the masked fractional peak enhancement map. In this example, the rendering circuitry 24 has rendered the masked fractional peak enhancement map using a shaded volume rendering (SVR) process, such as a global illumination (GI) process. The processing circuitry 22, e.g. display circuitry 26, is configured to display a 2D image representing the masked fractional peak enhancement map.

In this example, the region of interest comprises the vasculature of the brain of the patient or other subject.

The image shown in FIG. 12 may be understood as a composite image of the fames of any one of the image sequences described above. In this example, higher intensities represent later points in time relative to lower intensities. As described above, a value of each voxel of the peak enhancement map is represented by a respective time parameter. Each point in time represented by a time parameter in the fractional peak enhancement map is associated with a respective intensity value.

As can be seen from any one of FIGS. 8 to 12, the process described herein may allow for an angiogram-like visualisation, such as a digital subtraction angiography (DSA)-line visualisation, of the contrast material progression through the region of interest with an improved temporal resolution. For example, the process described herein may allow for the generation of an image sequence with improved temporal resolution based on the sets of acquired volumetric image data. This in turn may allow for the assessment of flow and/or a filling of vessels in the region of interest.

Certain embodiments provide a medical image processing apparatus comprising processing circuitry configured to acquire volumetric imaging data from a plurality of time points, obtain a vessel segmentation, estimate intermediate states based on intensities inside the vessels, and render these as a clinically relevant 2D video sequence.

A weighted temporal average map may be calculated.

The fractional delay may be used

$${f}_{d} = \frac{{\sum }_{p} {I}_{p} {t}_{p}}{{\sum }_{p} {I}_{p}} .$$

The fractional delay distribution may be analysed to find suitable windowing parameters.

M intermediate vessel segmentations may be generated through windowing of the delay map.

M volumes may be generated from original CT values, masked by intermediate vessel segmentations, and rendered (digital subtraction angiography (DSA), global illumination (GI) like).

M volumes may be generated from the fractional delay volume, and coloured to highlight early and late filling in renders.

The skewed temporal centre-of-mass may be used

$${f}_{d} = \frac{{\sum }_{p} {I}_{p}^{n} {t}_{p}}{{\sum }_{p} {I}_{p}^{n}}$$

for some exponent n. The delay map may be fed to a CNN trained (say on CT perfusion (CTP) data) to produce M vessel segmentations.

The visualisation may also highlight the surrounding tissue that would be perfused (be it a small shape region, MCA territory, or other region).

The frames corresponding to the acquired CTA scans (keyframes) may be indicated as such.

A split-sagittal view (e.g. that renders sagittal sequences for each hemisphere separately) may be rendered.

Sub-regions only of the vasculature (e.g. MCA regions) may be rendered.

It will be understood that references to a plurality of features may be interchangeably used with references to singular forms of those features, such as for example “at least one” and/or “each”. Singular forms of a feature, such as for example “at least one” or “each,” may be used interchangeably.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Whilst certain embodiments have been described, these embodiments have been presented by way of example only, and are not intended to limit the scope of the invention. Indeed the novel methods and systems described herein may be embodied in a variety of other forms; furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical image processing apparatus comprising processing circuitry configured to:
acquire a plurality of sets of volumetric image data of an anatomical region of a patient or other subject, each set of acquired volumetric image data corresponding to a respective different first point in time;
generate a segmentation of a region of interest of the anatomical region;
generate a plurality of sets of intermediate volumetric image data, one or more of the plurality of sets of intermediate volumetric image data representing the region of interest at a different second point in time, each second point in time corresponding to a point in time between two or more first points in time; and
generate an image sequence based on the plurality of sets of intermediate volumetric image data, the image sequence representing at least a part or all of the region of interest.

2. The apparatus of claim 1, wherein the processing circuitry is configured to generate the plurality of sets of intermediate volumetric image data based on voxel values of the plurality of sets of acquired volumetric image data.

3. The apparatus of claim 1, wherein at least one of the plurality of sets of intermediate volumetric image data represents the region of interest at a first point in time.

4. The apparatus of claim 1, wherein the processing circuitry is configured to:
generate an average of the plurality of sets of acquired volumetric image data, and to generate the segmentation of the region of interest of the anatomical region based on the average; or
wherein the processing circuitry is configured to generate the segmentation of the region of interest based on each of the plurality of sets of acquired volumetric image data.

5. The apparatus of claim 1, wherein the processing circuitry is configured to:
determine a plurality of time parameters, each time parameter being determined based on voxel values of corresponding voxels of the plurality of sets of acquired volumetric image data, each time parameter of the plurality of time parameters representing a point in time at which a value of the corresponding voxels of the plurality of sets of volumetric image data is likely to be maximum; and
generate peak enhancement volumetric data based on the plurality of time parameters.

6. The apparatus of claim 5, wherein the peak enhancement volumetric data comprises a plurality of voxels, a value of each of the plurality of voxels being represented by a respective time parameter.

7. The apparatus of claim 5, wherein the processing circuitry is configured to determine each time parameter (f_{d}) according to
$${f}_{d} = \frac{{\sum }_{p} {I}_{p} {t}_{p}}{{\sum }_{p} {I}_{p}} ,$$
wherein Iₚ corresponds to a voxel value at a respective first point in time tₚ.

8. The apparatus of claim 5, wherein the processing circuitry is configured to determine each time parameter (f_{d}) according to
$${f}_{d} = \frac{{\sum }_{p} {I}_{p}^{n} {t}_{p}}{{\sum }_{p} {I}_{p}^{n}} ,$$
wherein Iₚ corresponds to a voxel value at a respective first point in time tₚ, and n is an exponent.

9. The apparatus of claim 5, wherein the processing circuitry is configured to mask the peak enhancement volumetric data using the segmentation of the region of interest.

10. The apparatus of claim 5, wherein the processing circuitry is configured to at least one of:
determine a threshold based on the peak enhancement volumetric data;
apply the threshold to the peak enhancement volumetric data;
vary a value of the threshold; and
generate a set of segmentation volumetric data for each value of the threshold; or
wherein the processing circuitry is configured to apply a machine learning algorithm to the peak enhancement volumetric data to generate a plurality of sets of segmentation volumetric data.

11. The apparatus of claim 10, wherein the processing circuitry is configured to:
aggregate the plurality of sets of acquired volumetric image data to a single set of acquired volumetric image data and to mask the single set of acquired volumetric image data with at least one or each set of segmentation volumetric data; or
mask the average of the plurality of sets of acquired volumetric image data with at least one or each set of segmentation volumetric data to generate the plurality of sets of intermediate volumetric image data.

12. The apparatus of claim 1, wherein the processing circuitry is configured to render each of the plurality of sets of intermediate volumetric image data to generate a plurality of images representing at least the part or all of the region of interest, one or more of the plurality of images corresponding to a respective different second point in time.

13. The apparatus of claim 12, wherein the image sequence comprises the plurality of images.

14. The apparatus of claim 3, wherein the processing circuitry is configured to indicate an image in the image sequence as representing at least the part or all of the region of interest at a first point in time.

15. The apparatus of claim 12, wherein the region of interest comprises a first part and a second part and the processing circuitry is configured to render volumetric image data of each set of intermediate volumetric image data associated with the first part separately from volumetric image data of each set of intermediate volumetric image data associated with the second part.

16. The apparatus of claim 12, wherein the processing circuitry is configured to only render volumetric image data of each set of intermediate volumetric image data associated with a part of the region of interest.

17. The apparatus of claim 12, wherein the processing circuitry is configured to render each set of intermediate volumetric image data such that different parts of the region of interest are represented by different colours associated with different points in time.

18. The apparatus of claim 1, wherein the region of interest comprises at least one of:
an anatomical structure or a part thereof;
a region that surrounds the anatomical structure or a part thereof; and/or
a region or other anatomical structure adjacent to the anatomical structure or part thereof.

19. The apparatus of claim 1, wherein the anatomical region comprises at least one of:
a brain of the patient or other subject and the region of interest comprises at least a part or all of a vasculature of the brain; and/or
a liver of the patient or other subject and the region of interest comprises one or more vessels of the liver.

20. A medical image processing method comprising:
acquiring a plurality of sets of volumetric image data of an anatomical region of a patient or other subject, each set of acquired volumetric image data corresponding to a respective different first point in time;
generating a segmentation of a region of interest of the anatomical region;
generating a plurality of sets of intermediate volumetric image data, one or more of the plurality of sets of intermediate volumetric image data representing the region of interest at a different second point in time, each second point in time corresponding to a point in time between two or more first points in time; and
generating an image sequence based on the plurality of sets of intermediate volumetric image data, the image sequence representing at least a part or all of the region of interest.
